(19) United States
(12) Patent Application Publication
(10) Pub. No.: US 2011/0207079 A1
(43) Pub. Date: Aug. 25, 2011

(54) COATED SURGICAL AND DENTAL IMPLEMENTS AND IMPLANTS WITH SUPERIOR HEAT DISSIPATION AND TOUGHNESS

(76) Inventor: Jerry A. Rubin, Davenport, FL (US)

(21) Appl. No.: 13/015,422

(22) Filed: Jan. 27, 2011

Related U.S. Application Data
(60) Provisional application No. 61/298,832, filed on Jan. 27, 2010.

(51) Int. Cl. A61C 3/02; A61B 17/16; A61C 3/06; A61F 2/30; A61B 17/84; A61B 17/86

(52) U.S. Cl. 433/103; 606/176; 606/80; 433/166; 623/18.11; 606/300; 606/301; 427/2.29; 427/2.28

(57) ABSTRACT

The present application describes apparatus and methods for coating a base surgical or dental implement with a nickel boron or nickel phosphorous coating.

### CROSS-REFERENCE TO RELATED APPLICATIONS

The present application claims priority to U.S. Provisional Patent Application No. 61/298,832, filed Jan. 27, 2010; the contents of which are incorporated herein by reference in their entirety.

### FIELD OF THE INVENTION

Embodiments of the present invention relate, in general, to coated surgical and dental implements and implants with improved surface hardness and wear resistance, and decreased friction and galling. More particularly, embodiments of the present invention related to boron-based metal coatings on surgical and dental implements and implants.

### BACKGROUND OF THE INVENTION

In the following discussion certain articles, methods, patents, and publications will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Surgical and dental implements used for cutting, sawing, milling reaming or drilling into biological tissue (including bone) can generate significant amounts of heat between the tissue and the blade, bit, etc. This heat can cause damage to these biological tissues causing extensive cell damage or death. Further, such damage may render tissues more difficult to or unfit for transplantation or grafting. Moreover, the coefficient of friction for the surfaces of such implements can be high and could retard effective fluid flow.

For example, drilling reaming or milling of bone, cartilage, and other tissues is often desirable for medical purposes generally having to do with, but not limited to, the implantation of prosthesis, osteosynthesis screws or other elements on knees, hips, the spine and other bones or tissues. One of the most important applications is the drilling of the maxillary bone of a patient to prepare it for a dental implant in the fields of dental implantology and maxillofacial surgery. In such cases, the milling reaming or drilling procedure often involves the gradual drilling of the bone or other tissue through the gradual insertion of mill or drill bits or reamers of increasing diameters to form a cavity adapted to the dimensions of the implant or prosthesis. This involves rotating the tool, reamer or mill bit at the required speed. The exact rotation speed parameter is determined by a number of factors, mainly the geometrical characteristics of the mill bit used and the sequence of the milling process phases.

Mill and drill bits as well as reamers are available in a large number of shapes and sizes. This is mainly because each implant design usually comprises specific designs for specialized reamers, mill and drill bits to create cavities perfectly adapted to the dimensions of the implant. It is widespread practice during the milling, reaming, or drilling process to use bits or reamers with a range of different diameters for the same implant. This ensures that the bits or reamers used in each phase of the process adapt perfectly to the dimensions of the cavity to be formed. The speed is also indirectly affected by the heat generated in the procedure.

The wide variety of implants, bits and reamers used in conventional techniques means, therefore, that a broad range of milling, drilling, and reaming speeds (usually, but not limited to, from 800 to 1,500 rpm approximately) may be required. This high-speed milling or drilling causes both the bit and the bone tissue it operates on to heat up with the temperature of the bit often exceeding 40 degree C. Bone tissue cells are thermosensitive and have an optimal temperature of 37 degrees C. Any increase in this temperature can, therefore, be injurious to the cells and cause necrosis and/or burning of the surrounding bone tissues. Temperatures in excess of 56 degrees for one minute can even result in cell death, which unnecessarily adds to the trauma and suffering of the patient and can inhibit the desired healing of the bone and osseointegration of the implant. Rotation of the drill bit, mill bit, or reamer within the cavity being formed can also exert torque forces on both the tool and the surrounding bone tissues. Excessive torque forces and/or heat generation can possibly result in breakage of the tool within the cavity and, in some extreme cases, possible bone fracture. On the other hand, it is often desirable to perform the procedure quickly and efficiently to conserve time and minimize pain and suffering by the patient.

The thermal damage to which the tissue surrounding the implant is subjected during high-speed milling, reaming or drilling, to which the mechanical insult of the entire process must be added—has a damaging effect on the initial state of the cavity housing the implant. As a consequence, it takes longer for the bone to regenerate and bone-implant integration to occur, factors that impact the level of success of the operation.

It is, therefore, a widespread practice in conventional milling, drilling, and reaming techniques to apply a saline irrigation solution on the bit and the drilling area to prevent the bit and the surrounding tissue from heating up. This irrigation solution, however, can wash away signaling proteins and other soluble substances that play an active part in bone regeneration. These substances are released by the tissue in the area where tissue damage has occurred as a response to the damage and as a means of maintaining homeostasis, i.e., maintaining the biological and physio-chemical conditions prior to the damage, and are essential if the tissue is to rapidly recover. The specific physiological function of the signaling proteins is to transmit activation signals to the cell so that it can react to the deterioration suffered in the microenvironment. These proteins are connected to the extracellular matrix. This connection is broken when the tool impacts against the matrix. Many of these signaling proteins are characterized by their low molecular weight and their solubility. A saline irrigation solution easily dissolves and washes them away; therefore, stripping the tissue of the natural resources it uses to heal itself.

Furthermore, it is common practice to extract and collect the particles of tissue removed during milling, reaming or drilling and use them in autografting. Such a technique renders unnecessary the use of alternative and less preferred techniques marketed for this purpose such as allografting (homologous grafting of tissue obtained from a human tissue bank) or xenografting (the process of grafting tissue from one species of animal to another). As part of this process a suction device equipped with a filter collects all the bone particles. Following repeated analysis of high-speed milling, reaming, and drilling procedures, however, it has been found that the cells in these particles often die off as a result of the thermal and mechanical insults suffered during the process. Therefore it is desirable to develop a method of high speed drilling, reaming, and/or milling that is cooler without the necessity of applying fluid coolants or the ability to use far lower amounts of these coolants and, when desired, to collect the material removed for grafting.

Similar to the reamers, millers, and drill bits described above, dental and surgical burs are often used to drill into teeth or bone, and to shape internal and external tooth and/or bone surfaces. Because both tooth and bone materials are hard, a hard, sharp bur is needed to do the work. To perform the work with less trauma to the patient, such burs are often operated at very high speeds, and particular care is taken with lubrication and cooling. Still, however careful and advanced are the burs and the skills of the dentist or surgeon, the patient will be traumatized to some extent. Lowering this tendency requires attention to a variety of causes. While the improvement to each of these causes may individually be very small, the cumulative effect to the patient may be very important.

To the dentist or surgeon, the cost of burs is important, and the need to change them during a procedure because they become dull is a decided nuisance. Any enhancement of durability then has two aspects. One is that a bur which remains sharp longer can in the long run be less traumatic. Another is that a bur which is likelier to remain optimally sharper for a longer period of time and therefore be less expensive. Moreover, such an instrument could not only remain sharp longer, but in many cases would cut faster than prior art devices.

As a consequence of this invention, drilling and grinding speeds can be shortened, vibration effects from a non-uniformly worn bur can be reduced, and to these improvements can additionally be added improved lubricity and heat transfer properties. A problem that is inherent in conventional efforts to make a sharp and durable bur is that these materials tend to be brittle, and crack and break. Breakage during usage represents a danger to the patient. A piece of the bur may come loose and not be readily retrievable, or may jam in a tooth or bone crevice, requiring sacrifice of some of the surrounding tissue. These tendencies may be countered not only by maintaining sharpness longer, but also by providing toughness as well. With conventional burs, these objectives are counter to one another. This invention enables them to be obtained or at least approached at the same time.

While efforts have been made to accomplish the above objectives, the existing burs generally involve composite structures in which cutting bodies are bonded or embedded in a substrate. An example is tungsten carbide cutting inserts bonded to a stainless steel substrate. Such an arrangement provides multiple opportunities for failure. Optionally an entire bur might be made of tungsten carbide to resist the tendency of the bur to fragment, but this is relatively expensive, and if used should have a means to extend its useful life.

As another example, diamond coated burs have diamond crystals embedded in a relatively soft substrate. When the substrate erodes, the crystals are lost, and the bur no longer functions. Depositing a diamond surface on the tool to protect the substrate lengthens the life of the bur. Thus, the prior art recognizes the value of providing a more durable dental bur, which also has improved inherent lubricity and heat conductivity.

It is also common in the surgical arts where cutting, sawing, grinding, polishing, drilling, milling, reaming, or other similar implements are used, to employ a guide or guard to ensure that the cut, bore, grind, etc. is in the proper location and does not extend to adjacent areas of tissue. Frequently, however, the surgical implement may come in contact with the guard resulting in friction, heating and even galling of the implement.

For the above reasons the prior art has recognized a need for cooler functioning surgical implements that do not heat up or gall when they contact adjacent tissue or surgical guards or guides. Surgeons utilize blades, bits, etc. of various shapes and configurations in the performance of surgical procedures. For example, the powered saw is an important powered tool that a surgeon employs to perform certain surgical procedures. A typical powered saw has a handpiece in which is housed either an electrically or pneumatically driven motor. The motor is attached, through a drive shaft, to a head. The head is designed to removably receive a saw blade. Actuation of the motor causes movement of the saw blade. This movement of the saw blade gives the blade the power to cut through the tissue it is employed to separate. Powered surgical saws are able to cut through both hard and soft tissue much faster, and with greater accuracy, than the manually operated saws that they have replaced. The high speed of operation, however, results in many of the problems (i.e., heating, galling) discussed previously.

Most surgical saw blades are categorized as either reciprocating or oscillating. An oscillating blade, when connected at its proximal end to a hub of a powered surgical saw, pivots about the hub such that the distal end reciprocates in an arc-like manner. An example of such an oscillating blade may be had by reference to U.S. Pat. No. 5,135,555. Often, such oscillating blades are used in conjunction with a cutting guide to provide for a precision cut. The cutting guide commonly has a head of some type with a slot therethrough having side walls. As the surgeon cuts along the slot with the oscillating blade, the sides of the blades can violently hit against the side walls of the guide causing the lateral side walls of the blades to deform or mushroom out. The mushrooming of the blades' sides can cause the blade to rub against the slot, thereby increasing friction, which in turn can cause a higher torque on the powered instrument. Increased friction can also increase the heat and debris at the resection site. Further, with excessive mushrooming the blade could jam within the cutting guide.

Another type of saw blade is known as a reciprocating saw blade and is designed to interact with a precision slot which guides the saw blade in reciprocating movements to cut tissue (including bone tissue) during the performance of surgery. Customarily, a reciprocating surgical blade has a plurality of teeth on each of its two edges. These teeth are customarily provided in a set, that is, consecutive teeth are bent in alternate directions. As this set is provided on known surgical saw blades, the ends of the teeth extend upwardly and downwardly beyond the respective planes of the respective side faces of the blade. Thus, often, it is these ends of the teeth which guide the saw blade within the precision slot. Of course, inherently, this causes wear of the teeth quite prematurely and results in the surgeon having to change saw blades quite often during the surgical case.

Yet another type of saw blade is known as a sagittal saw blade and consists of a flat elongated blade with a proximal saw engaging slot and a distal transverse surface having a plurality of teeth thereon, with the teeth being flat in configuration, which is, not provided in a set. These are but examples of surgical saw blades which may form a part of the present invention.

As an example, surgical saws like those described above are utilized in the course of many various procedures to resect or cut anatomical tissue including bone. For example, during the performance of total knee replacement surgery, several bone cuts are made in the knee to prepare, contour or shape the knee to receive a prosthesis. Five cuts are generally made in the distal femur, i.e., transverse distal femoral cut, anterior femoral cut, posterior femoral cut and anterior and posterior chamfer cuts. One cut is usually made in the proximal tibia, i.e., transverse proximal tibial cut, and another cut is usually made in the patella to remove the patellar facets. Such bone cuts are typically made with an end cutting oscillating saw having a flat saw blade driven to sweep back and forth, up to thousands of times per minute, while being advanced into the bone. To accurately resect the bone to fit the contours of the prosthesis, cutting blocks or guides are usually attached to the bone and used to guide the saw blade to establish the proper orientation for the bone cuts. Conventional cutting guides commonly have slots through which the saw blade is inserted, the slots having sufficient clearance to allow the saw blade to oscillate while maintaining a close enough fit with the saw blade to guide the saw blade to obtain the proper bone cut. Illustrative of oscillating saw blades for resecting bone are U.S. Pat. No. 4,513,742 and U.S. Pat. No. 4,584,999 to Arnegger U.S. Pat. No. 5,087,261 to Ryd et al, U.S. Pat. No. 5,263,972 and U.S. Pat. No. 5,439,472 to Evans et al, which also disclose power drive systems for driving the saw blades, and U.S. Pat. No. 5,306,285 to Miller et al. Representative of cutting guides or blocks for guiding end cutting oscillating saw blades to form bone cuts are U.S. Pat. No. 4,892,093 to Zarnowski, U.S. Pat. No. 5,092,869 to Waldron and U.S. Pat. No. 5,178,626 to Pappas.

Conventional oscillating bone saws and guide systems, therefore, have many disadvantages including excessive vibration of the bone saws resulting in inaccurate bone cuts and poor surface finishes, elevated temperatures of the bone saws due to friction between the saw blades and the cutting blocks and/or between the saw blades and the bone, thermal-necrosis of the bone due to the high temperatures caused by friction, the inability to use cement-less prostheses where healthy bone is damaged from high temperatures, production of metal debris from contact of the saw blades with the cutting blocks, consumption of an exorbitant amount of power by the drive systems for the bone saws, the need for larger, more expensive batteries due to the high power requirements of the drive systems, excessive noise generated by the drive systems, increased risk of contamination to medical personnel from blood-borne and other pathogens carried by anatomical tissue and fluids that are splattered, atomized or comminuted by the oscillating saw blades, and difficulties in guiding the oscillating saw blades.

Studies have been conducted on the effect on bone tissue of blades which have been galled for whatever reason. In article titled “Orthopedic Saw Blades a Case Study” by H. W. Wevers, et al, published in the Journal of Arthroplasty, Volume 2 No. 1, March 1987, this problem is discussed. The following is quoted from this article: “Because these blades are used primarily for total knee arthroplasty, it is probable that the damage occurred from direct contact of the cutting edges with metal templates or instruments used in the operation. This type of damage had a direct influence on the mechanical work needed to operate the saw.” Later in the publication, the following is stated: “Excessive heat induces thermal damage to osteocytes and expands the zone of necrosis beyond that shown microscopically.” Further, the following is stated: “Smooth, accurately cut surfaces are recognized as an important factor for bone ingrowth into porous-coated prostheses. Such clean bone cuts enhance prosthetic fit and setting, therefore promoting an even load bearing to the bone, and improved alignment of the prostheses or osteotomies.” Finally, the following is stated: “Damage to blade cutting surfaces due to inadvertent contact with templates and instruments may be unavoidable with currently available techniques.”

Further, a publication titled “Avoiding Thermal Damage to Bone: Machining Principals [SIC] Applied to Powered Bone Surgery a Literature Review”, by Ray Umber, et al. further discusses the problems attendant in the prior art. The following is disclosed therein: “Thus, cutting with a dull tool, increases the amount of frictional heat generated, much of which is now located in the workpiece itself. With a dull tool not only is the surface of the workpiece increasing in temperature but also the cut is no longer clean.”

A further problem with prior art saw blades is also set forth in this publication. In particular, prior art saw blades are so designed that it is difficult to provide water to the site of the operation for cooling purposes and to remove bone chips which are generated during sawing. Due to present saw blade design, “coolant can not reach the dissection site”.

In recognizing these problems, people have responded by providing surgical saw blades with a hard coating bonded thereto which will protect the blade surface to thereby increase the lifespan of the blade. It is also known in the industry to apply a coating to the exterior of a blade to reduce galling of the blade during use. However, mushroomed edges scraping against a cutting guide can scrape off the gall-resistant coating along the edge. For example, others have attempted to coat surgical saw blades with polytetraflurorethylene, better known by the trademark TEFLON. Experiments with TEFLON coated blades have revealed increased efficiency with marked reduction in galling. However, it was found that autoclaving as well as use of the blades in a precision slot would result in the coating stripping off and it was further discovered that chunks of coating would sometimes enter the surgical site. These chunks were very offensive to the surgical site since it was found that the TEFLON flakes were not bio-compatible with the tissues of the patients.

Others have experimented with ceramic coatings such as calcium phosphate and aluminum oxide but these have similarly failed for several reasons. Firstly, the melting point of the ceramics is higher than that of the blade material and, as such, during a thermal spray process, the temper of the blade is destroyed. Further, a blade coated with ceramic cannot be bent or flexed without cracking the coating.

Similarly others have experimented with combinations of methyl methacrylate and ceramics. While these combinations are superior to the TEFLON, calcium phosphate and aluminum oxide, adherence to the metallic blades is inferior. Even the milling of grooves to hold the methyl methacrylate and ceramics to the blade does not solve the problems in a satisfactory manner.

The above examples referencing saw blades are provided for illustrative purposes and it will be obvious to one skilled in the art that similar problems are present with many current art surgical implements such as, but not limited to, drills, milling devices, burs, shears, etc. The problems with heating, galling and distortion of the implement are common across all these devices. Thus the prior art show that there is a need for coatings for surgical implements that reduce heat buildup (and the accompanying cell damage and death), adhere strongly to the implements and provide a level of flexibility without damage to the coating material.

Another problem with prior art surgical and dental implements is that they generally are not sufficiently durable to permit multiple cycles of reprocessing. Reprocessing is a technique whereby used surgical or dental implements that have become too dull or worn to function effectively are reconditioned such that they are again suitable for use. All types of cutting, reaming, drilling, milling and abrading tools are subject to medical reprocessing including, but not limited to, scalpels, surgical saw blades, surgical and dental burs, drill and mill bits, reamers, surgical shavers, etc. The F.D.A., however, sets strict standards for reprocessed medical and dental implements. Typically prior art implements like those discussed here are not sufficiently durable to withstand multiple cycles of reprocessing while still meeting F.D.A. standards. In fact, only about 50% medical blades (for example) are reprocessed more than once. Because reprocessed surgical and dental implements are functionally equivalent to but cost much less than new implements, their use results in substantial savings to hospitals, doctors, dentists and others who use them. Thus it would be desirable to develop surgical and dental implements that are sufficiently durable to withstand more cycles of reprocessing before they must be discarded.

As referenced above, the surgical guides, guide blocks, and guards often contribute to heating and galling when surgical implements such as, but not limited to, surgical saws come in contact with them. These problems are exacerbated by the fact that the surgical implement must often work in close proximity to the guides, guide blocks, and guards. Excessive distance between the guides will result in increased deflection and increased sawing, drilling or milling variation which is extremely undesirable in a surgical procedure where even minor deviation from the intended point of penetration can be devastating. Inadequate distance between the guides and the implement however will cause the guide and implement to heat and wear excessively. The prior art has tried to use different materials to make the guide pads, blocks and guards. Materials such as steel or other metals are undesirable due to the inherent friction and galling created by the metallic implements rubbing against them. Others in the art have tried, Teflon pads, and ceramic guide pads but this approach is unsuccessful either because these coating material are not sufficiently durable or they do not adhere well to the guides, guards, and blocks.

Another embodiment of the present invention relates to orthopedic implant devices including but not limited to replacement joints, bone screws, and other implantable orthopedic hardware. Generally, titanium and titanium alloys are used for orthopedic applications because of their strength, corrosion resistance, and biocompatibility. The tribological behavior of titanium and its alloys, however, is characterized by a high coefficient of friction and poor wear performance, resulting in a tendency for titanium and its alloys to seize or gall under conditions of wear. Therefore, in those orthopedic applications requiring enhanced wear resistance properties, the surface of a titanium implant must be hardened. In the past, surface hardening of orthopedic implants has been achieved either by depositing a nitride coating on the surface of an implant, or by forming a layer of titanium nitride (TiN) on the surface of a titanium substrate.

A TiN layer is produced on the surface of a titanium implant by various nitriding methods, including gas nitriding, chemical salt bath nitriding, plasma or ion nitriding, and ion implantation. Of these alternatives, gas nitriding is believed to be the earliest method used for hardening titanium, and still exhibits advantages over the other methods in terms of cost and ease of manufacture. For instance, gas nitriding permits efficient batch processing of many parts concurrently in a furnace chamber; whereas, the plasma nitriding and ion implantation methods require line-of-sight bombardment of the workpiece, thereby limiting the number of parts that may be processed concurrently.

Gas nitriding of titanium and its alloys has historically been performed at elevated temperatures in the range of 700 degrees C. to 1200 degrees C. (1292 degrees F. to 2192 degrees F.) U.S. Pat. No. 4,768,757 discloses a method for nitriding the surface of a titanium dental cast, wherein it is stated that the temperature generally used for the nitriding treatment falls in the range of 700 degrees C. to 880 degrees C. because nitriding generally begins to proceed in the neighborhood of 700 degrees C. and the heat distortion or phase transition point of titanium is about 882 degrees C. Characteristic of virtually all gas nitriding processes is the formation of a relatively thick TiN layer on the surface of the titanium, caused by a scaling reaction. Essentially, successful nitriding of a titanium orthopedic implant for the purpose of providing a hardened surface is defined by the observance of a distinct and measurable TiN layer achieved by elevated temperatures, as taught by the prior art.

It has now been discovered that the aforementioned gas nitriding process, as applied to a titanium orthopedic implant device, may produce several undesirable changes in the physical and mechanical properties of the device. Notwithstanding increases in overall surface hardness, the TiN layer formed on the surface of the device by gas nitriding at elevated temperatures tends to be brittle and exhibits increased surface roughness, both of which cause losses in the fatigue strength of the implant. Also, temperature induced changes in the dimensions of the titanium orthopedic implant device may occur.

Potential losses in the fatigue strength and increases in the surface roughness of a titanium orthopedic implant device are of particular concern in orthopedic applications involving load bearing prostheses in articulating contact with bone or polymers. For instance, under conditions of sliding or articulation of the nitrided implant against other surfaces, particularly bone and polymers, the increased surface roughness may produce wear debris that can act as an abrasive medium. Consequently, it is desirable to reduce the possibility of wear debris and its potential impact on the stability of orthopedic implants by enhancing the surface hardness of the titanium material without substantial losses in fatigue strength or wear resistance properties.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 shows an exemplary test platform, according to one embodiment.

FIG. 2 shows a before and after image of an uncoated blade with multiple teeth.

FIG. 3 shows a before and after image of one tooth on an uncoated blade.

FIG. 4 shows a before and after image of a coated blade with multiple teeth.

FIG. 5 shows a before and after image of one tooth on a coated blade.

FIG. 6 is a graph comparing average temperatures of coated and uncoated blades.

FIG. 7 is a graph comparing maximum temperatures of coated and uncoated blades.

FIG. 8 is a graph comparing cut times for coated and uncoated blades.

### DETAILED DESCRIPTION OF THE PREFERRED EMBODIMENT

Embodiments of the present invention relate to surgical and dental implements including, but not limited to, surgical saws, saw blades, slitting blades, cutting blades including scalpels, shears, reamers, drill and mill bits, (including those used in the field of dentistry) polishing/grinding discs, and dental/surgical burs, surgical guides and guards. In other embodiments, the present invention relates to orthopedic implant devices including, but not limited to, replacement joints, bone screws, and other implantable orthopedic hardware.

In one embodiment, the surgical and dental implements and orthopedic hardware of this invention have a nickel boron coating applied from a bath containing metal salts, reducing agents, chelating agents, and stabilizers. The source of the metal stabilizer is usually, but is not limited to, a metal compound or salt that is used to stabilize the nickel boron bath. The amount of the metal stabilizer in the coating is about 0% to about 4% by weight of the coating, preferably about 0.5% to about 1.0%, most preferably about 0.5%. The coating composition can range from about 1-8% by weight of boron, preferably about 5-7% and most preferably about 6.0-6.5% boron. The balance of the coating is preferably nickel. The co-deposited metal stabilizer is considered to form an alloy deposit of nickel and boron with the metal stabilizer element. Metal stabilizers such as thallium sulfate, lead tungstate, or lead chloride can be used.

There are three main types of alkaline electroless nickel boron coatings. The Type-1 coatings are taught in U.S. Pat. No. 3,674,447 to Bellis filed Jul. 4, 1972, and U.S. Pat. No. 6,183,546 to McComas filed Nov. 2, 1998 which are incorporated by reference. Metal elements such as thallium and lead are used to stabilize the electroless plating. These metal elements form an alloy with the nickel boron coating. A typical Type-1 electroless coating has about 1-3% by weight thallium, preferably about 1.5-2.5%, most preferably about 2%, about 1-4% by weight boron, preferably about 2.5-3.5%, most preferably about 3% and about 92-97% nickel, preferably about 93-96%, more preferably about 94-95%, most preferably about 94.5% by weight.

The Type-2 is taught in U.S. Pat. No. 6,066,406 to McComas which is incorporated by reference. A typical Type-2 electroless coating has about 1-8% by weight boron, preferably about 5-7%, and most preferably about 6.0-6.5%, about 0-2% by weight lead, preferably about 0.5-1.0%, and the balance being nickel.

The Type-3 is taught in U.S. patent application Ser. No. 10/903,687 filed Aug. 2, 2004 entitled Electroless Coating with Nanometer Particles to Ed McComas. A typical Type-3 electroless coating has nickel boron plus nanometer diamond-like carbon (DLC) particles, about 1-8% by weight boron, preferably about 5-7%, and most preferably about 6.0-6.5%, about 0-2% by weight lead, preferably about 0.5%, and balance being nickel and co-deposition of solid DLC particles.

In another embodiment, the surgical and dental implements and orthopedic hardware of this invention have a nickel phosphorus (Ni—P) coating. Such a coating may be applied via an electroplating method as taught in U.S. Pat. No. 6,099,624 to Martyak or by an electroless method as taught in U.S. Pat. No. 3,953,624 to Arnold, both of which are herein incorporated by reference. Ni—P comes in three main types, “low P” with about 1-3% phosphorus, “mid P” with about 5-8% phosphorus, and “high P” with about 10-13% phosphorus, by weight. Brighteners can be added to make them shiny or left out if a dull finish is acceptable.

In one non-limiting embodiment of the invention, the implement is a surgical saw blade coated with one of the above disclosed coatings. In another non-limiting embodiment of the invention, the implement is a surgical or dental reamer, drill or mill bit or a dental or surgical bur with one of the above disclosed coatings. In yet another non-limiting embodiment the implement is a guide, guide block or guard used to aid in accurately cutting, drilling, milling, reaming or otherwise penetrating a specific surgical site coated with one of the above disclosed coatings. These embodiments of the invention are not only unexpectedly more durable in use, but unexpectedly reduce heat generation in adjacent tissue while also minimizing the necessity for coolants and flushing water in the surgical site

For example, a blade for a powered surgical saw (e.g., a reciprocating blade adapted for use in performing a median sternotomy) coated with one of the above described nickel boron or nickel phosphorus coatings results in an unexpected and dramatic decrease in the overall temperature at the incision site as compared to previous art blades. Consequently there is less cell damage and dramatically reduced need for cooling fluids at the surgical site. Further, the saw's power requirements are unexpectedly reduced and the surgeon may act with greater precision as the frictional and torque forces acting back on the saw are greatly reduced. Similar unexpected results may be obtained when these coatings are applied to the other types of surgical and dental implements described in this application like drills, milling devices, reamers, burs, grinding discs, etc.

Some of the advantages of the present invention are that friction between the surgical implement and a guide or guard employed at the surgical site is unexpectedly minimized, friction between the implement and the tissue (including bone tissue) is also unexpectedly minimized, and operating temperatures of the implement are unexpectedly relatively lower than conventional implements lacking the disclosed coating. This provides particular benefit in the realm of orthopedic surgery and dentistry because thermal-necrosis of the tissue is reduced and possibly avoided allowing the use of cement-less prostheses or implants. Also, decreased friction between the implement and surrounding tissue and/or guards results in more efficient cutting, reaming, drilling and/or milling with less wear. Thus, the cutting, reaming, drilling or milling implement can be operated at lower speeds and/or reduced power requirements as well as friction and the implement can be more precisely guided during cutting and undesired cutting of soft tissue is avoided.

Another advantage of the invention as applied to reaming, milling, drilling or cutting procedures where bone tissue is involved is that it protects the tissue surrounding the reaming, drilling, milling or cutting area as much as possible thereby preventing the area from heating up and, at the same time, negating the secondary effects derived from the use of saline irrigation solution as a coolant—mainly the washing away of the intrinsic cellular signals that help the tissue heal more quickly and become biologically stronger.

Yet another advantage of the invention as applied to reaming, milling, drilling or cutting procedures is that it unexpectedly results in greater efficiency in clearing bone and tissue debris from the surfaces of the dental or surgical implement. For example, because of the superior lubricity and reduced surface friction resulting from the disclosed nickel boron or nickel phosphorus coatings, such debris do not build up on the surface or in between the teeth of surgical saw blades to the same degree that they would on prior art blades. Likewise, debris build up is limited to a great extent within the flutes of a reamer, drill bit, or mill bit when coated with one of the disclosed nickel boron or nickel phosphorus coatings. This can result in a cleaner cut or penetration with less effort and more precision.

Yet another advantage of the invention as applied to reaming, milling, drilling or cutting procedures is that it may be possible to make these surgical and dental implements lighter, and/or thinner. This is so because of the superior wear resistance, hardness, and toughness imparted to these implement when coated in accordance with the teachings of this disclosure. Such an implement may require less base material to create a tool that is as, or more durable and functional than prior art devices.

Yet another non-limiting embodiment of the invention is a process for surface hardening an orthopedic implant device including, but not limited to, replacement joints, bone screws and other orthopedic fasteners made of pure titanium or a titanium alloy, wherein the surface hardness of the device is enhanced while increasing wear resistance and fatigue strength by applying one of the above disclosed nickel boron or nickel phosphorus coatings. The invention also encompasses said orthopedic implant devices in accordance with the claimed process.

An advantage of the surface hardening method of the present invention is that the surface of an orthopedic implant device made of titanium is hardened without substantially affecting the mechanical and physical properties of the material.

Another advantage of the surface hardening method of the present invention is that the method is adapted for use on load bearing prostheses that contact with bone or polymers, due to an significant improvement in wear resistance coupled with a minimal loss in fatigue strength. This is a somewhat complex process. For example, the mating material in the wear couple with Ni—B or Ni—P, the loads chosen, the lubrication used, and the alloys coated by Ni—B or Ni—P are the variables that determine improvements to wear and fatigue loss.

A further advantage of the surface hardening method of the present invention is that it permits batch processing of orthopedic implant devices without requiring a “line of sight”, as opposed to prior art plasma nitriding processes requiring a “line of sight” to the parts for ion bombardment. The nickel boron and nickel phosphorous surface hardening methods employ chemical baths into which devices to be coated are immersed. In most cases so long as the device/component surfaces are wet by the chemistry it will be coated. In plasma processes devices/components are put into a vacuum chamber, temperatures are increases, and a plume of ions travels from a source to the component by line-of-sight trajectory. The plasma process is therefore limited in its ability or incapable of uniformly coating complex geometry.

The invention, in one form thereof, provides a method of manufacturing an orthopedic implant device having enhanced surface hardness and wear resistance properties. The method includes several essential steps, including an initial step of providing a titanium substrate in the form of an orthopedic implant device, or component thereof. A surface hardening step is then performed. Specifically, the surface of the titanium substrate is hardened with a nickel boron or nickel phosphorous coating by the process as detailed above.

Consider, for example a replacement hip joint or other similar prosthesis. The mammalian hip consists of a ball portion on the femur bone and a corresponding socket in the hip. When one or both of these portions is damaged a replacement joint made from titanium may be the best option to restore mobility to the victim. The replacement joint will have a similarly structured ball and socket configuration which inevitably involves contact between the ball surface and the socket surface that must move relative to each other. If these surfaces are unnecessarily rough there may be an unacceptable level of friction and heat buildup in the joint resulting in discomfort for the patient. Likewise if the joint material or its surface coating is brittle, even normally acceptable levels of friction between the two pieces can result in failure of the joint. Under this embodiment of the invention, however, the contacting joint surfaces (and possibly other surfaces) are coated with a nickel boron or nickel phosphorus coating resulting in superior wear resistance and lubricity. Thus, such a joint will wear better, last longer and create fewer problems for the recipient. While, a replacement hip joint is cited here as an example, this embodiment is not limited to such. One skilled in the art will immediately recognize that other types of orthopedic implants including, but not limited to, replacement knee joints, elbow joints, shoulder joints, etc. will similarly benefit from the coatings described above. Coatings may also be applied to stationary parts such as bone screws, etc.

In another non limiting embodiment of the invention the disclosed coatings are used on the blades of surgical shavers. Such a tool typically consists of a motorized handpiece attached to a spinning curved blade housed within a protective sleeve although those skilled in the art will recognize that other configurations are possible. Such shavers are commonly used, for example, in arthroscopic surgery to shave and/or shape cartilage and soft tissue.

This embodiment of the invention is advantageous because the coating increases the wear resistance of the shaver blade such that it remains sharp longer. This results in less frequent changing of the shaver blade which is both costly and time consuming. Further, the superior heat dissipation properties of the disclosed nickel boron or nickel phosphorus coating, as discussed elsewhere in this specification, result in less tissue damage when employed for surgical purposes.

As has been discussed above, all of the disclosed embodiments, along with other embodiments that will be obvious to those skilled in the art, offer substantial, unexpected benefits over prior art devices. One such benefit common to all these embodiments is that of superior wear resistance. In the case of cutting, drilling, reaming, milling, abrading, or shaving type implements this means that the tool will retain its cutting or abrading surface properties longer resulting in longer life and more effective use. Those skilled in the art will also recognize that this will increase the reprocessability of these implements. A more durable implement will withstand more reprocessing cycles while still meeting FDA regulations. In effect the cutting, drilling, reaming, milling abrading or shaving surfaces of these implements may withstand more reprocessing cycles where by they are returned to a serviceable condition. This results in a lower overall cost by allowing the cost of these implements to be amortized over more surgical or dental procedures. Additionally, because dentists and surgeons can bill for their procedures independent of the equipment costs, greater reprocessability may result in higher profit margins for these providers.

### Experiments

To illustrate the above advantages, the following tests were conducted. It is understood that these test procedures and the results therefrom are intended as examples only and in no way limit the scope of the present invention.

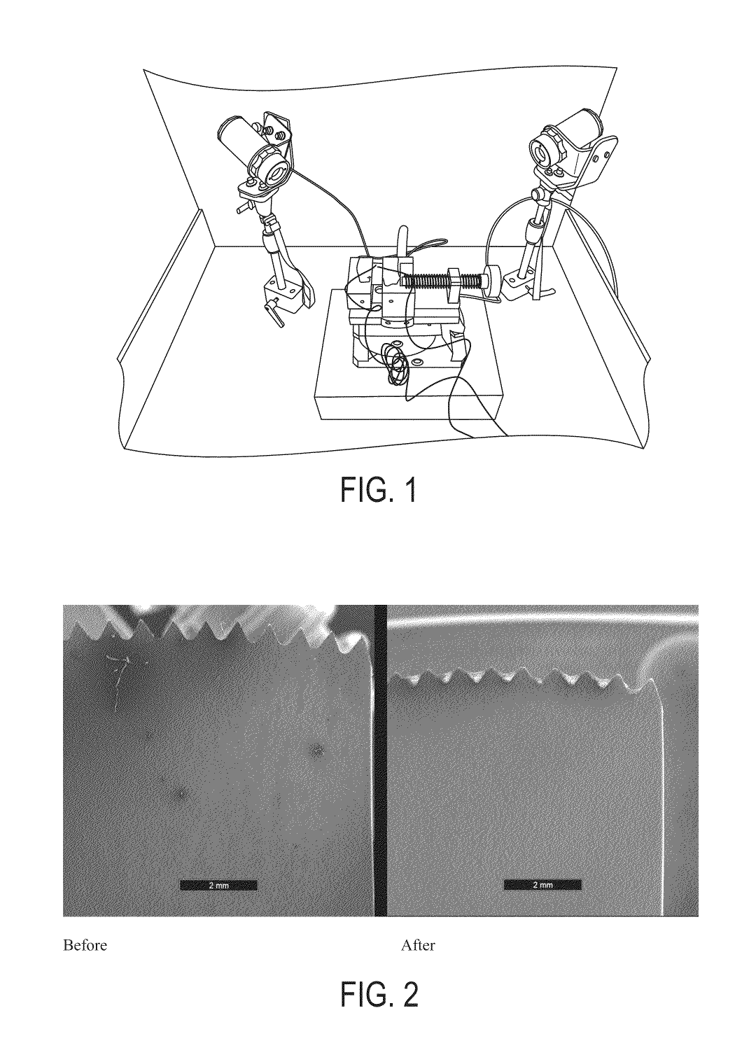

First, a test platform (FIG. 1) was constructed of an aluminum table standing 30 inches high with a 33 inch wide×24 inch deep smooth surface, a 24 inch tall back plate and two 4 inch tall side plates. In the center of the table surface there were two holes to align and mount a vice. The custom-made vice had jaws with 1×2¾ inch flat-faced surfaces that open to 3 inches and compress to fully closed using a ¾ inch diameter screw. The vice was mounted onto an aluminum block that has two alignment pegs to mount onto the table surface. The vice contained three load sensors (Loadstar Sensors, Fremont, Calif., 94538) calibrated from 0-15 pounds using NIST traceable equipment. The load sensors were incorporated in the vice assembly and export readings to a log file through LoadVUE software (Loadstar Sensors, Fremont, Calif., 94538) in pounds of force in three orientations plus the total load in the downward direction at a rate of 0.1 Hz. The device allowed accurate readings of applied downward force whether the force acts on the center of the vice or on an object cantilever-mounted in the vice. A FLIR Systems (North Billerica, Mass., 01862) ThermaCAM™ SC640 radiometric infrared imaging camera was used to collect real-time temperature data of the system (saw and bone) and the MR ExaminlR software was used to analyze the thermographic data to determine average and maximum temperature versus time along the cut groove. The camera temperature range used was 0° C. to 500° C. with an accuracy of ±2° C. or 2% of the reading. Measured temperatures ranged from ambient (approximately 25° C.) to near 200° C. A Stryker Instruments (Kalamazoo, Mich. 49001) TPS Sagittal saw system was used at 50% power to cut the bone. Cuts were made at a 45° angle from vertical through the cortex of the bone to the cancellous material. Cuts took from three to twenty five seconds, depending on the type of saw blade used, the bone properties, and the use and rate of water irrigation. Saw blades are either Ni—B coated (to 0.0005″ thickness) or as-received MicroAire (Charlottesville, Va. 22911) SP-125 small oscillating saw blades, measuring 9 mm×31 mm×0.4 mm. Irrigation was delivered through MasterFlex® L/S® 13 tubing clamped approximately 2 inches above the cut and optimally delivered to the back side of the blade to reduce interference of the radiometric measurement. Irrigation flow was controlled by a MasterFlex® peristaltic pump model 77800-60 (Cole-Parmer Instrument Company, Vernon Hills, Ill., 60061) operating at 0.75 to 2.0 ml/min. Fresh bovine back rib bones were used as cutting substrates and are cleaned of soft tissues and membranes before use. Bones typically measured 180×25×13 mm and were cantilever-mounted in the vice so that the cut is made at least 100 mm from the edge of the vice and perpendicular to the major axis of the bone. The cortex was typically 5 mm thick and the cancellous material 3 mm in diameter at the thickest part of the bone. Following the above procedure several trials were made using both the coated and uncoated blades.

Qualitatively, the coated blade showed clearly superior results. When the uncoated blade was used, visible smoke was generated. Further, the tool and blade became too hot to handle. Conversely, when the coated blade was used there was no visible smoke, the tool and blade ran much cooler and the cuts were much faster.

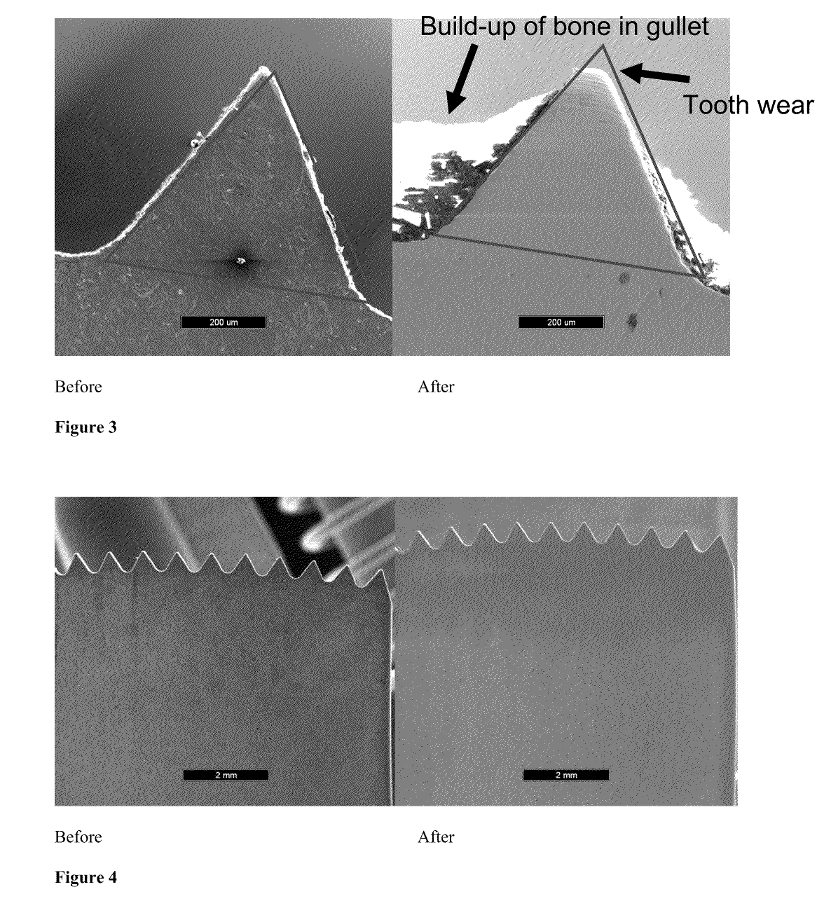

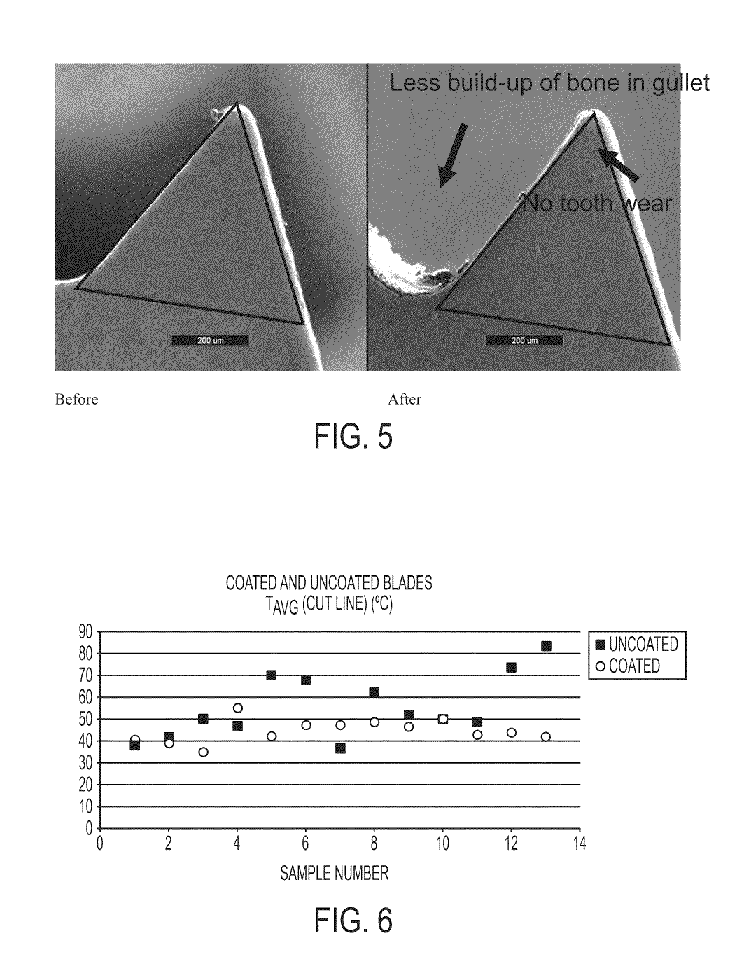

Quantitatively the results obtained from cutting the bovine bone are illustrative. Wear on the uncoated blade was evidenced by clear rounding of the blade's teeth after a single cut as can be seen in FIGS. 2 and 3. Further, FIG. 3 shows that there was a significant buildup of bone matter between the blade's teeth when the uncoated blade was used. Conversely, no wear was evident on the coated blade's teeth after an identical test and there was significantly less buildup of bone matter on the blade (see FIGS. 4 and 5).

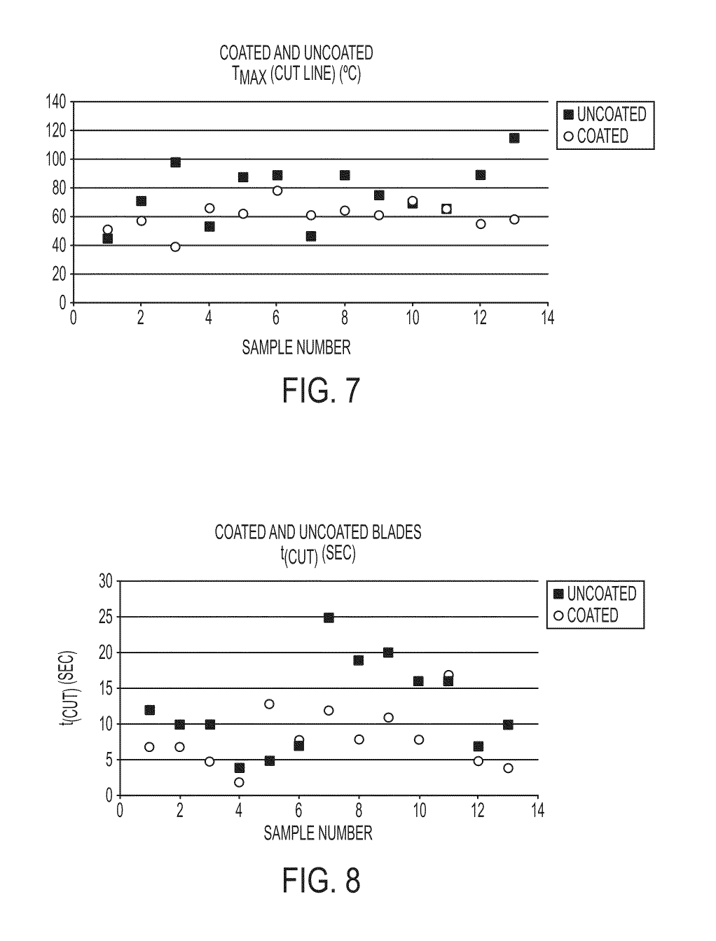

Additionally, the quantitative data confirmed the qualitative assessments that the coated blade operated cooler and quicker. Identical tests according to the above procedures were performed 13 times each with a coated and non coated blade. The average temperature recorded for all tests using the coated blade was 45 degrees C., while the average temperature for the uncoated blade was 55 degrees C. The spread was even more pronounced when maximum temperatures were considered. The maximum temperature recorded for any test using the coated blade was 61 degrees C., while the maximum temperature for any test using the uncoated blade was 15 degrees higher at 76 degrees C. The complete data can be seen in FIGS. 6 and 7. Similarly, the cut time was, on average lower for the coated blade than for the uncoated blade (see FIG. 8 for full data). Thus, the preceding test data shows that coated blades offer superior wear performance, operate at a lower temperature and reduce cutting time when compared to uncoated blades.

In addition, the residual bone dust on each blade was tested to determine whether there was any residual metal debris from the blades in the dust. During an actual surgical or dental procedure it is normally desirable to limit contamination of the surrounding tissue with metal debris from the surgical or dental implement being used. To perform the test, an energy dispersive X-ray analysis (EDS) technique was used. In such a test, a sample is exposed to an electron beam inside a scanning electron microscope (SEM). These electrons collide with the electrons within the sample, causing some of them to be knocked out of their orbits. The vacated positions are filled by higher energy electrons which emit x-rays in the process. By analyzing the emitted x-rays, the elemental composition of the sample can be determined. Thus far, only one such test has been conducted and the results obtained are preliminary, however, they are quite promising.

Analysis of the dust on the uncoated stainless steel blade revealed two particles of copper and five particles comprised of iron, chromium and nickel in ratios consistent with that in stainless steel. In addition one of the stainless steel particles was quite large, measuring nearly 100 microns. By way of contrast, no nickel-boron particles were found in the residual dust on the coated blade and only one small (˜10 micron) particle of stainless steel was found. Because there were no nickel-boron particles found in the debris, it is unlikely that the lone stainless steel particle found on the coated blade came from the blade itself since the coating would have to be completely abraded before the stainless steel underlying it could be exposed. Therefore, it is likely that the lone stainless steel particle found on the coated blade came from somewhere else, perhaps from the saw body or the test platform. In any case, the results of this test show a dramatic decrease in the residual metal debris present when the coated blade was used. Thus, the coated blades tested are clearly superior to the prior art blades.

It is noted that, while the above tests were conducted using coated and uncoated MicroAire SP-25 oscillating saw blades, the results are merely illustrative of similar results that would be obtained using other types of surgical and dental implements. It will be immediately apparent to those skilled in the art that the mechanical forces acting on the saw blades in the above tests would be similar in nature and degree to those acting on other types of surgical and dental implements under similar test condition and, therefore, similar results would be expected.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents.

1. A surgical or dental apparatus comprising:
a base surgical or dental implement; and
a nickel boron or nickel phosphorous coating on the base surgical or dental implement.

2. The apparatus of claim 1, wherein the coating has a composition comprising about 1-3% by weight thallium, about 1-4% by weight boron, and about 92-97% by weight nickel.

3. The apparatus of claim 1, wherein the coating has a composition comprising about 1-8% by weight boron, about 0-2% by weight lead, and about 90-99% by weight nickel.

4. The apparatus of claim 1, wherein the coating has a composition comprising about 1-8% by weight boron, about 0-2% by weight lead, and about 90-99% by weight of nickel and solid nanometer diamond-like carbon particles that are co-deposited.

5. The apparatus of claim 1, wherein the coating has a composition comprising nickel and about 1-3% by weight, about 5-8% by weight, or about 10-13% by weight phosphorus.

6. The apparatus of claim 5, wherein the coating further comprises one or more brighteners.

7. The apparatus of claim 1, wherein the base surgical or dental implement is selected from the group consisting of: a surgical saw blade, a surgical or dental reamer, a surgical or dental drill or mill bit, a dental or surgical bur, a surgical or dental grinding disc, a surgical or dental guide, guide block or guard, and combinations thereof.

8. The apparatus of claim 1, wherein the base surgical or dental implement is selected from the group consisting of: a replacement joint, a bone screw, an orthopedic fastener, and combinations thereof.

9. The apparatus of claim 1, wherein the base surgical or dental implement comprises titanium or titanium alloy.

10. A method of coating a surgical or dental apparatus, the method comprising:
providing a base surgical or dental implement comprising a region for accepting a coating;
providing a bath comprising one or more metal salts, one or more reducing agents, one or more chelating agents, and one or more stabilizers;
immersing the region for accepting a coating in the bath; and
forming a coating on the region for accepting a coating.

11. The method of claim 10, wherein the one or more stabilizers is a metal or salt.

12. The method of claim 10, wherein the one or more stabilizers is present at about 0%-4% by weight of the coating.

13. The method of claim 12, wherein the one or more stabilizers is present at about 0.5%-1.0% by weight of the coating.

14. The method of claim 13, wherein the one or more stabilizers is present at about 0.5% by weight of the coating.

15. The method of claim 12, wherein the balance of the coating is nickel.

16. The method of claim 10, wherein the one or more stabilizers are selected from the group consisting of: thallium sulfate, lead tungstate, lead chloride, and combinations thereof.

17. The method of claim 10, wherein the coating comprises about 1-8% by weight of boron.

18. The method of claim 17, wherein the coating comprises about 5-7% by weight of boron.

19. The method of claim 18, wherein the coating comprises about 6.0-6.5% by weight of boron.

20. The method of claim 17, wherein the balance of the coating is nickel.
